(19) United States
(12) Patent Application Publication
(10) Pub. No.: US 2023/0277747 A1
(43) Pub. Date: Sep. 7, 2023

(54) APPARATUS AND METHODS FOR POINT-OF-CARE RED BLOOD CELL WASHING

(71) Applicant: Hanuman Pelican, Inc., New Orleans, LA (US)

(72) Inventors: Randy DORIAN, San Diego, CA (US); Michael D. LEACH, Warsaw, IN (US); Richard W. STORRS, Berkeley, CA (US); Scott R. KING, New Orleans, LA (US)

(73) Assignee: Hanuman Pelican, Inc., New Orleans, LA (US)

(21) Appl. No.: 18/177,047

(22) Filed: Mar. 1, 2023

Related U.S. Application Data
(60) Provisional application No. 63/268,810, filed on Mar. 3, 2022.
(60) Provisional application No. 63/269,222, filed on Mar. 11, 2022.

(51) Int. Cl. A61M 1/36; A61M 1/34

(52) U.S. Cl. CPC A61M 1/3693; A61M 1/34

(57) ABSTRACT

Apparatus and methods for point-of-care red blood cell washing are disclosed. One method of filtering a suspension of red blood cells may generally comprise introducing a wash solution to the suspension of red blood cells such that a combined volume is retained within a first compartment, applying a sweeping action upon the volume, applying a pressure upon the volume while applying the sweeping action such that the volume is urged through a filter membrane at a predetermined filtration rate, maintaining the filtration rate over a predetermined period of time such that hemolysis is maintained below a threshold level, and collecting the volume within a second compartment.

### CROSS-REFERENCE TO RELATED APPLICATIONS

This application claims the benefits of priority to U.S. Prov. Apps. 63/268,810 filed Mar. 3, 2022 and 63/269,222 filed Mar. 11, 2022, each of which is incorporated herein by reference in its entirety.

### FIELD OF THE INVENTION

The present invention relates to apparatus and methods for the filtration and the centrifugation for processing of blood components. More particularly, the present invention relates to apparatus and methods for effectively filtering and centrifuging red blood cell components.

### BACKGROUND OF THE INVENTION

Rejuvesol® (Citra Labs Biomet, Warsaw, Ind.) is presently the only FDA-approved product to rejuvenate stored red blood cells (RBCs) which restores 2,3-Disphosphoglycerate (2,3-DPG) and ATP to levels in fresh blood or higher. The product has the ability to potentially reduce the number of units required by hospitals by improving the functionality of the transfused units. Rejuvesol® is comprised of Pyruvate, Inosine, Adenine, and Phosphates (“PIPA” formulation) and all of these ingredients provide raw materials for the biochemical synthesis of 2,3-DPG and ATP to occur when incubated, recommended for 1 hour at 37° C.

Inosine, the active agent in Rejuvesol® solution, is metabolized to hypoxanthine by red cells that in turn is administered to patients. In patients, hypoxanthine is metabolized to xanthine by xanthine oxidase, then to uric acid. Chronic elevation of serum and urine xanthine and uric acid levels can result in renal calculi (nephrolithiasis).

Following Rejuvesol® treatment the RBCs are washed to reduce residual inosine prior to red cell transfusion. However analyses have identified elevated levels of hypoxanthine, an inosine metabolite, in the supernatant of the red cells following Rejuvesol® treatment and incubation. This is also reduced by washing.

RBCs treated with Rejuvesol® must be further processed (washed or cryopreserved and deglycerolized) prior to transfusion to remove rejuvenation-derived purines, inosine and hypoxanthine. Glycerolized, cryopreserved RBC also require washing, even without the addition of Rejuvesol®. Thus, a device for washing RBCs may have utility for this application as well.

Approved rejuvenation techniques include mechanical cell washing using existing cell washing devices. However, recent research suggests that mechanical cell washing (with inherently significant shear) has formerly underappreciated adverse effects on red cells. In the REDJUVENATE Trial (conducted by the University of Leicester) manual washing was used after evaluation of several mechanical cell washers. Manual washing was found to be the most operationally practicable way of supplying red cells for this clinical study and was therefore performed in validation studies. The efficacy of this technique for the removal of inosine and hypoxanthine, and the risk of subsequent lysis or cell free hemoglobin release had not been evaluated thus far and was therefore specifically addressed in the validation study. The vast majority of Rejuvesol® treated blood units in the US are washed with blood washers or cell salvage devices.

The manufacturing process employed in clinical evaluation also included a manual washing step, whereas the U.S. Food and Drug Administration approved process uses a mechanical cell washer. Administration of inosine with transfused red cells is considered as a potential risk to patients and the effectiveness of manual washing on inosine removal has not been demonstrated thus far.

Accordingly, a Point-of-Care device is desirable which would allow for removing the Blood Bank from the process flow and would make the product more widely available and more rapidly accepted in the marketplace. The device should be able to achieve an acceptable level of reduction in extracellular solutes (notably inosine and hypoxanthine) while causing minimal hemolysis, should be rapid and simple to operate and reasonable in cost. Minimal transfer steps are desirable to simplify the process and importantly to reduce the chance of microbial contamination.

### SUMMARY OF THE INVENTION

The washing step of the point-of-care device includes a first aspect of a filtration process and a second aspect of a centrifugal process. One method of filtering a suspension of red blood cells may generally comprise introducing a wash solution to the suspension of red blood cells such that a combined volume is retained within a first compartment, applying a sweeping action upon the volume, applying a pressure upon the volume while applying the sweeping action such that the volume is urged through a filter membrane at a predetermined filtration rate, maintaining the filtration rate over a predetermined period of time such that hemolysis is maintained below a threshold level, and collecting the volume within a second compartment.

The second aspect of washing involves a centrifugal process. One method for centrifuging a suspension of red blood cells may generally comprise introducing the suspension of red blood cells within a reservoir such that the suspension drains from the reservoir and into a pack compartment circumferentially positioned about the reservoir, rotating the suspension within the circumferential pack compartment such that a wash solution within a wash compartment flows radially outward and up through the pack compartment and into a supernatant compartment, and stopping a rotation of the suspension such that waste from the supernatant compartment drains into a waste compartment and an RBC pack remains within the pack compartment.

One variation of an apparatus for centrifuging a suspension of red blood cells may generally comprise a reservoir positioned within a housing and located along an axis of rotation, wherein the reservoir defines a port for receiving the suspension of red blood cells, a wash reservoir located below around the reservoir, a pack compartment located circumferentially about the reservoir and in fluid communication with the wash reservoir through a fluid lumen extending from a top portion of the wash reservoir and a bottom portion of the pack compartment, a supernatant compartment in fluid communication with the pack compartment and located circumferentially between the wash reservoir and the pack compartment, and a waste reservoir located below the wash reservoir and in fluid communication with the supernatant compartment.

### BRIEF DESCRIPTION OF THE DRAWINGS

FIG. 1 illustrates a perspective assembly view of one variation of a filtration chamber.

FIG. 2 illustrates a graph showing the results of filtering a volume of red cells using a 9 cm diameter polycarbonate track etched membrane disk filter.

FIG. 3 illustrates a graph of results when a dilute RBC suspension was filtered through a 70 mm 0.4 micron PETE membrane.

FIG. 4A illustrates an example in a graph of the total wash volume/supernatant volume plotted over volume per wash against the residual fraction extracellular solutes.

FIG. 4B illustrates another example in a graph illustrating the relationship shown above in FIG. 4A of the number of fractions against the residual solute.

FIG. 5A illustrates an example in a graph showing the filtration over time.

FIGS. 5B and 5C illustrate the resulting fluid filtered with the different membranes.

FIG. 6 illustrates an example in a graph showing that the filtration rate and hemolysis with the Sterlitech 1μ PETE membrane.

FIG. 7 illustrates an example in a graph showing filtration parameters using a 4 cm radius 1 micron GVS PCTE membrane.

FIG. 8 illustrates the several collected filtrate fractions.

FIG. 9A illustrates a perspective view of a planetary red cell washer which closely emulates the hand wash method in a fully automated process within a hermetically sealed rotor.

FIG. 9B illustrates a partial cross-sectional top view of the system in which the rotor may be comprised of concentric compartments.

FIG. 9C illustrates a side view of the system showing the relative positioning of the compartments.

FIGS. 10A and 10B illustrate perspective and top views of another variation of a system which eliminates the need for valves.

FIGS. 10C and 10D show additional cross-sectional and perspective cross-sectional views of the rotor.

FIG. 11 illustrates a cross-sectional side view of another variation.

FIGS. 12A-12E illustrate an example of a sequence of steps that may be used in the centrifugal process without the use of valves.

FIGS. 13A-13C illustrate perspective, cross-sectional perspective, and cross-sectional side views of another variation of the system.

FIGS. 14A and 14B illustrate perspective and cross-sectional perspective views of yet another variation of the system having one or more vanes through which the wash solution may be redirected through channels within the vanes.

FIG. 15 shows a cross-sectional side view of the rotor and the various compartments.

FIGS. 16A-16E illustrate another example of a sequence of steps of how the RBC may be washed in the centrifugal process.

FIGS. 17A and 17B illustrate side and cross-sectional perspective vies of another variation of the system having separated compartments.

FIGS. 18A-18F illustrate another example of a sequence of steps of how the RBC may be washed in the centrifugal process.

FIG. 19 illustrates a schematic view of one variation of an assembly for washing RBCs.

FIG. 20 illustrates a graph illustrating the RBC sedimentation over time when washed with a rotor system.

### DETAILED DESCRIPTION OF THE INVENTION

Filtration

As part of the washing step of a point-of-care device, a first aspect involves a filtration process and a second aspect of a centrifugal process. The first aspect of the filtration process is discussed below.

The Hemosep device (Advancis Surgical, United Kingdom) is a bag with two compartments: a first compartment holds blood or a suspension of red cells which is separated by a filter made of a porous membrane from a second compartment which contains an absorbent. The absorbent sucks out liquid and the filter holds back cells. The membrane pore size is less than 5 microns and preferably between 1 and 2 microns.

Washing by filtration is possible but with the Hemosep device, once supernatant fluid has been absorbed into the absorbent, the process is finished and further washing (such as a second wash step using the same device) cannot be done. Moreover, even if all of the supernatant fluid is removed from a suspension of red cells so that the red cells are close packed, the remaining volume of interstitial fluid is between 3-6% of the pack volume so that at best, only 94-97% of extracellular material trapped between the cells can be removed by this process in a single step. Achieving even this level of reduction is unlikely because red cells at high concentration comprise a thick slurry which cannot be driven to sufficiently sweep the membrane surface to prevent clogging without causing significant hemolysis.

Substituting vacuum (or pressure) for the absorbent might be used. Another possibility is to use centrifugation to provide the driving force. A very gentle driving force and also a very gentle sweeping of the membrane may be utilized. For instance, slowly varying a low speed spin rate could provide a low pressure and a very gentle sweeping. Alternatively, pressure may be used as a driving force with orbital shaking to provide a sweeping of the membrane to prevent clogging of the pores and a representative example is provided herein.

While a vacuum (low pressure) can be applied downstream of the membrane to drive the fluid through the filter membrane, a gentle pressure may instead be applied upstream as the driving force.

To allow continuous or multiple cycles of batch washing of red cells, a red cell suspension may be contained in a chamber fitted with a porous membrane. The compartment may be rigid or flexible so long as the membrane is rigidly supported. Red cells are introduced into the compartment and wash may be added through a feed line which may include a valve to control batchwise addition of wash solution. The addition of wash solution may be by gravity feed, applied pressure, or vacuum (on the outflow side of the filter) or by pumping, etc. The compartment containing the cells may be mounted, e.g., on an orbital shaker, to provide for sweeping of the membrane. Employing this means of sweeping rather than circulating the cell suspension through a connected fluid circuit may avoid the need for a pump (e.g., a peristaltic pump), which could cause damage to the red cells. Transmembrane pressure differential is preferably sufficient to provide a filtration rate permitting removal of extracellular solutes to a sufficient extent within a reasonable time frame. For example, through multiple batch washes, better than 99% of extracellular solutes can be removed in less than, e.g., an hour, and preferably better than 99.9% in less than, e.g., a half hour. The greater the reduction in the least time is generally better. Hemolysis should be less than, e.g., 0.8% and preferably less than, e.g., 0.3%, and most preferably under, e.g., 0.1%. Ideally, the hematocrit of the red cells after washing is complete should be between, e.g., 50-65%.

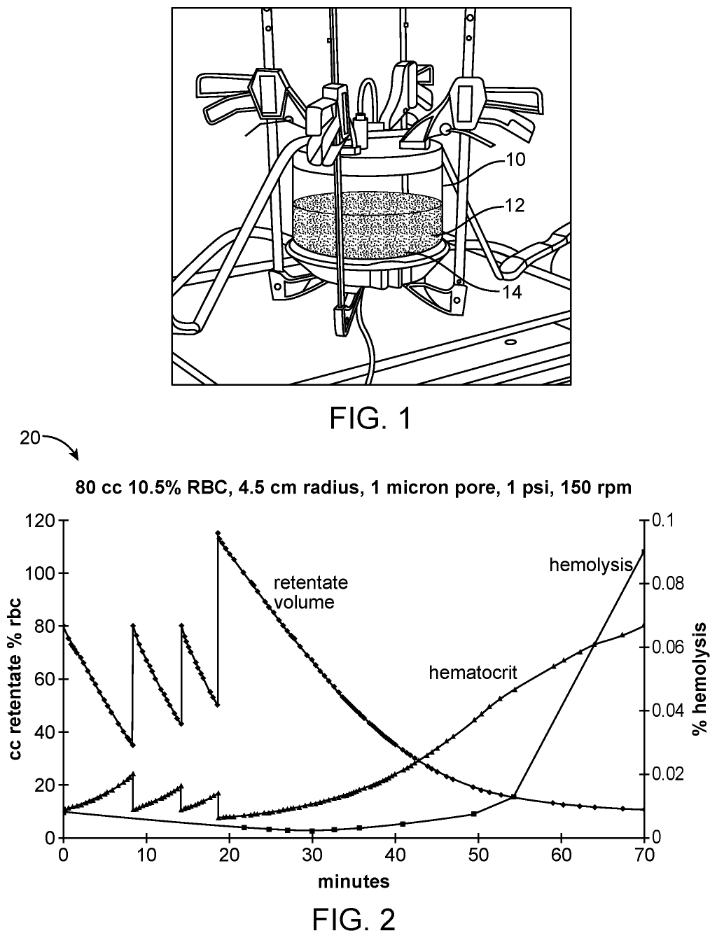

To test the feasibility of this approach, a membrane supported by a screen was installed in a pressure chamber 10 into which red cells 12 and wash solution were introduced and several different types of membrane were tested, as illustrated in the perspective assembly view of FIG. 1. Promising results were attained using track etched membranes 14 (e.g., polyethylene or polycarbonate), which may have smooth surfaces and uniform pore sizes. Alternative variations may incorporate non-uniform pore sizes. The chamber was mounted on an orbital shaker which was rotated at 150 rpm (with an amplitude of about 1 cm) to provide sweeping. The chamber was pressurized to force fluid to flow through the membrane 14 and out through a drain line beneath the supporting screen.

Multiple wash cycles can reduce the time required to remove contaminating extracellular solutes while maintaining a low level of hemolysis. For example, starting at around 10% hematocrit and concentrating to around 50%, then diluting back to 10% and filtering again down to 50%, filtration is relatively rapid and hemolysis is minimal. Extracellular solute concentration by these two steps may be potentially reduced by 99%. A third wash may bring the contaminant removal to 99.9%. In the assembly depicted in FIG. 1, filtration was allowed to proceed for a while, then additional wash solution was added and filtration resumed. This was repeated a few times. It can be seen, as shown in the graph of FIG. 2, that with a 90 mm diameter polycarbonate track etched membrane disk with 1 micron pore size, it takes roughly 30 minutes to concentrate 80 cc 10% RBC to 50% at 1 psi with hemolysis less than 0.02%. Scaling up to a 250 cc unit of 80% RBC (200 cc RBC) and a one-foot diameter membrane, at 1 psi it would take about an hour for each cycle. At 3 psi hemolysis is still minimal and it should take less than 30 minutes per cycle or around an hour for 2 cycles, achieving a 99% reduction in contaminants (consuming 4 liters of wash solution). Increasing the membrane diameter to a foot and a half would bring the time down to about ½ hour.

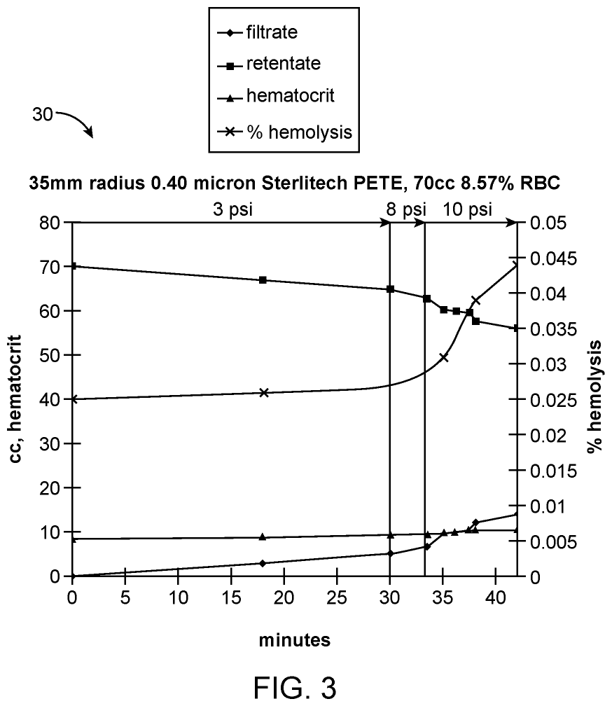

FIG. 3 illustrates a graph 30 of results when a dilute RBC suspension was filtered through a 70 mm (effective diameter) 0.4 micron PETE membrane (Sterlitech Corp., Auburn, Wash.), starting at 3 psi transmembrane pressure differential which yielded good results with the 1 micron PCTE membrane (GVS S.p.A., Italy). The filtration rate was relatively slow, so pressure was increased to 8 psi and then 10 psi. The filtration rate at the higher pressures was faster than at 3 psi, but still about 10 times slower than with the 1 micron membrane (GVS S.p.A) at 1/10 the pressure. Hemolysis was substantial even at low hematocrit.

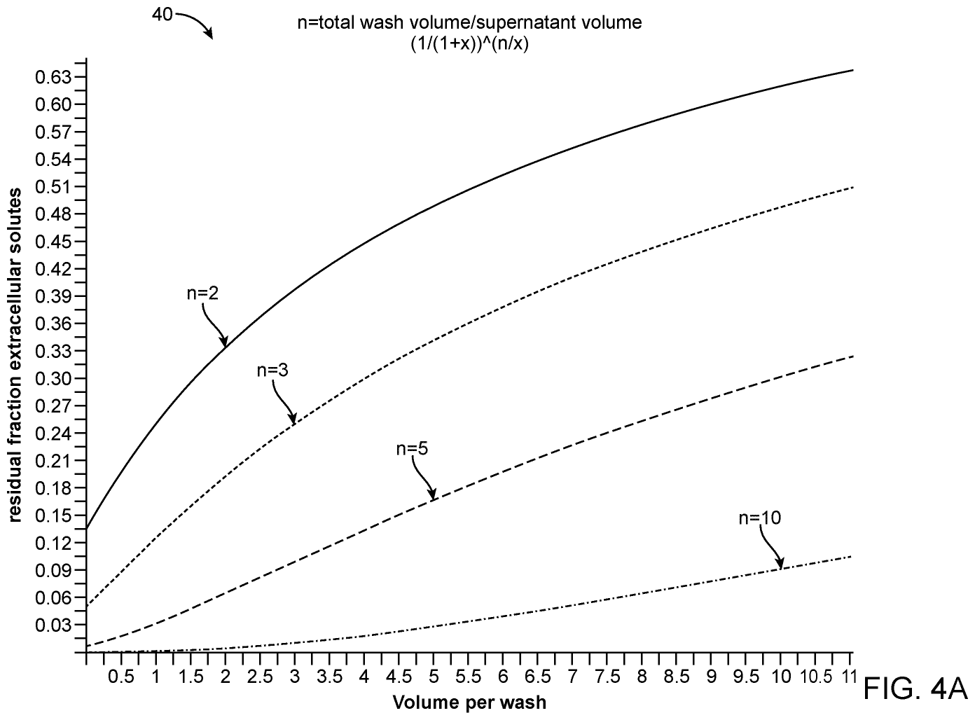

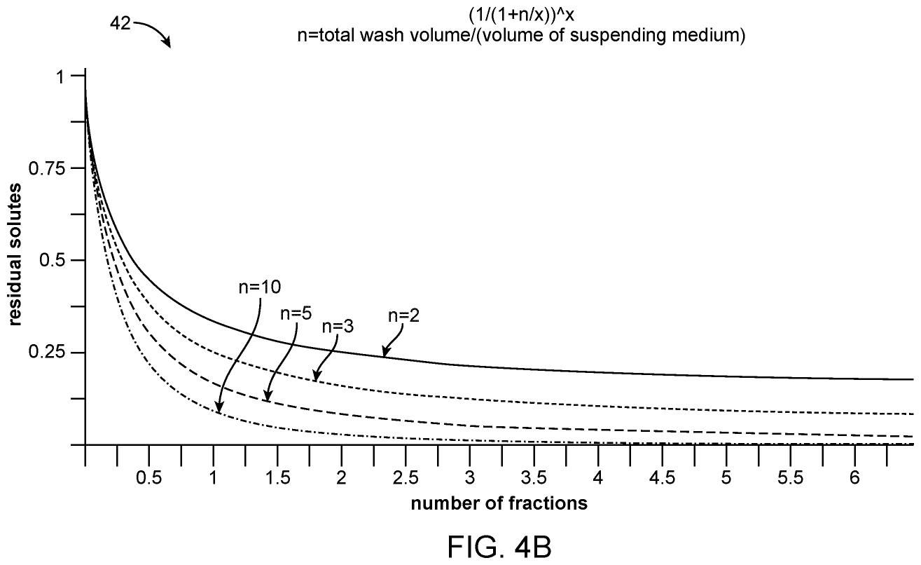

FIG. 4A illustrates an example in a graph 40 of the total wash volume/supernatant volume plotted over volume per wash against the residual fraction extracellular solutes where n is equal to the total wash volume/supernatant volume. For removing extracellular solutes, 2 washes with ½ volume are better than 1 wash with 1 volume. Four washes with ¼ volume are better still. For any given total volume of wash to be used, the more the wash volume per wash is broken down and increase the number of washes accordingly, the better. At the limit, we get the best washing by breaking down the wash volume infinitely and doing an infinite number of washes using infinitesimal volume per wash, e.g., continuously adding wash at the same rate as filtrate is removed.

At 50% hematocrit, we get decent filtration rates with modest hemolysis. Avoiding higher hematocrits during filtration to minimize RBC damage is generally desired. If the hematocrit is held at 50% by making the filtration compartment capacity twice the volume of packed RBC while continuously adding wash solution, the maximum wash efficiency can be attained without severe damage to the cells.

If hemolysis proves unacceptably high with continuous filtration at 50% hematocrit, some efficiency can be sacrificed by maintaining the hematocrit at a lower level by increasing the volume capacity of the compartment, increasing the total wash volume accordingly if necessary to increase extracellular solute removal, and then filter out a little more volume to raise the final hematocrit.

The intercept of the curves at the ordinate axis represents the amount of residual extracellular solutes after filtering 10 times the packed RBC volume from continuously fed compartments with various fixed volumes (in the graph, volumes of 1+(10/n) times the packed RBC volume).

FIG. 4B illustrates another example in a graph 42 illustrating the relationship shown above in FIG. 4A of the number of fractions against the residual solute where n is equal to the total wash volume/volume of suspending medium. The approximate limits where n=2 is 14%; where n=3 is 5%; where n=5 is 0.7%; and where n=10 is 0.005%.

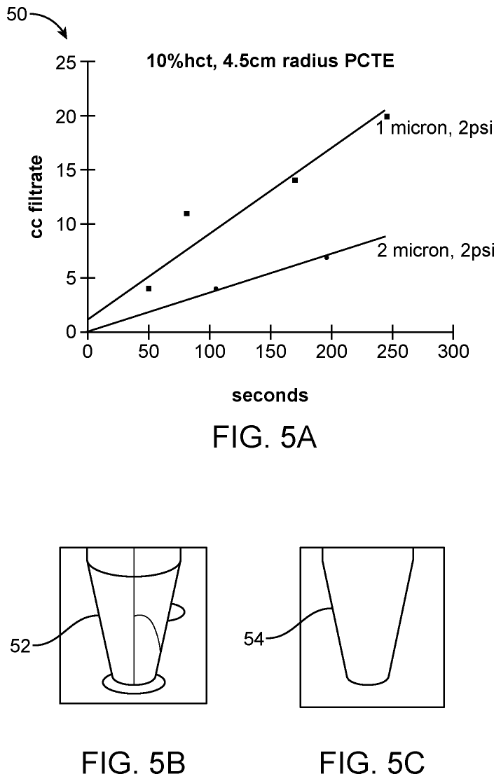

FIG. 5A illustrates an example in a graph 50 showing the filtration over time. As shown, the filtration rate was lower and hemolysis higher with a 2 micron PCTE membrane (GVS S.p.A) compared with a 1 micron GVS PCTE membrane both under a pressure load of 2 psi. FIG. 5B illustrates the resulting fluid 52 with the 1 micron GVS PCTE membrane which was filtered more effectively and with lower hemolysis compared to the resulting fluid 54 illustrated in FIG. 5C with the 2 micron PCTE membrane filtered less effectively and with higher hemolysis.

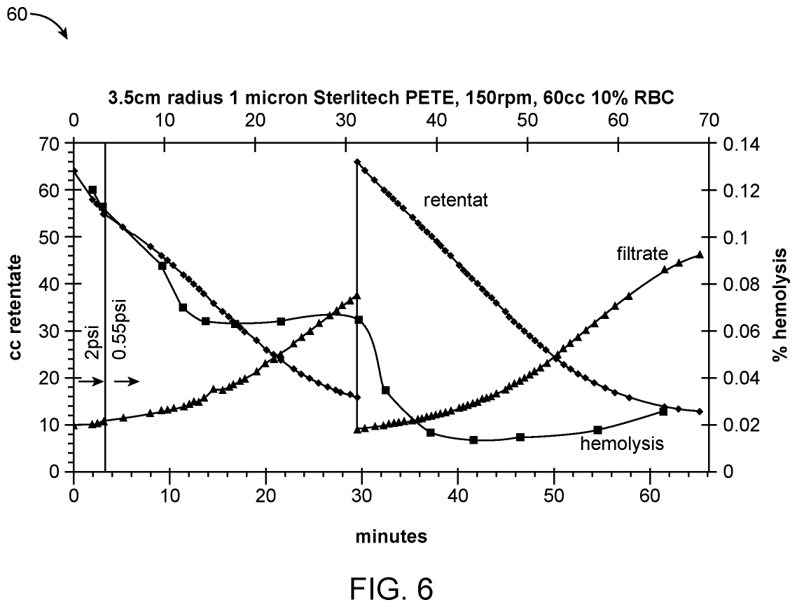

FIG. 6 illustrates an example in a graph 60 showing that the filtration rate and hemolysis with the Sterlitech (Auburn, Wash.) 1μ PETE membrane were comparable to those of the GVS 1μ PCTE at significantly lower transmembrane pressure differential. At higher pressures, hemolysis was significant. Either 1μ PETE or PCTE would be suitable to reduce the level of extracellular solutes in a unit of RBC by 99% in 2 wash cycles in about a half hour using an 18 inch diameter membrane.

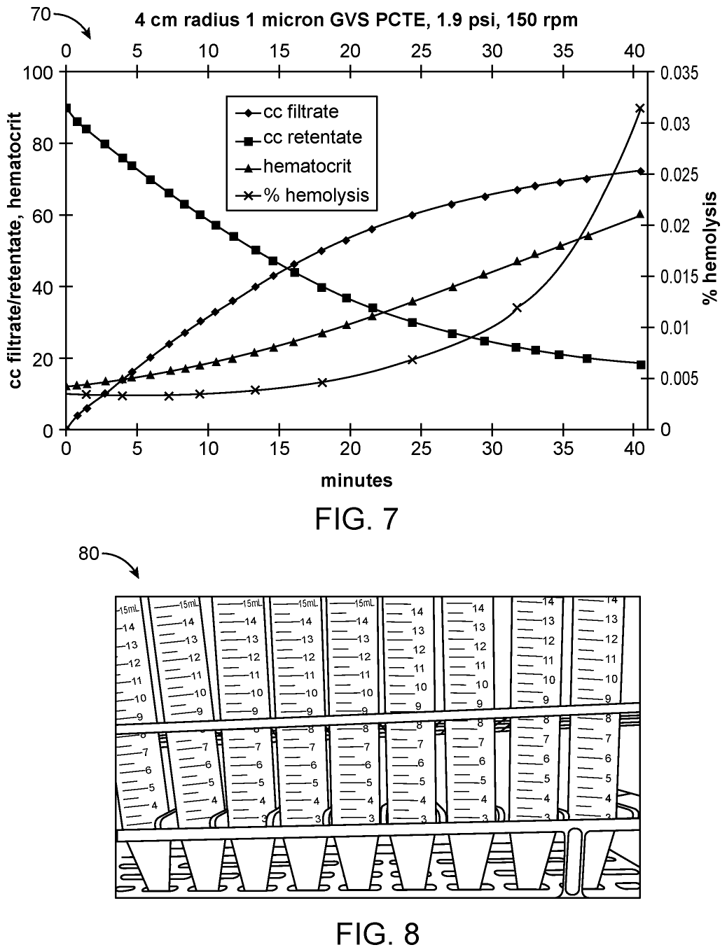

A volume of 90 cc 12% RBC was filtered to a final hematocrit of 60%, as shown in the graph 70 of FIG. 7. Hemolysis at the end was about 0.03%. The time required was 40 minutes. Scaling up to a 200 cc unit of RBC (17× volume) with a 12 inch diameter filter (14× area), filtration time would be about 50 minutes. Increasing the filter diameter to 18 inches, the time would be reduced to about 20 minutes. Increasing the pressure from 2 psi to 3 psi, the time should theoretically be reduced to about 15 minutes. A single filtration step would reduce extracellular solutes by about 92%. Redilution and refiltration would result in a 99.3% removal, requiring about a half hour to complete. A higher rate of hemolysis and a lower final hematocrit can be tolerated as a workable system. FIG. 8 illustrates the several collected filtrate fractions 80 in various collection tubes.

Centrifugation

Turning now to the second aspect of the washing step of the point-of-care device, the centrifugal process is discussed below with respect to several different variations in which any one variation or any one variation incorporating any number of different features between different embodiments may be utilized.

Generally, a volume of blood may be introduced into a RBC reservoir within the device along with a volume of wash solution. The device may combine the volume of blood and wash solution for spinning in a centrifugal motion to wash the RBCs contained within the volume of blood. In one example, if a volume of RBC introduced into the device is set a minimum of 200 mL, a target purity of 99.7% can be achieved by running the RBC through six cycles of washing using one liter of wash solution over a process time of less than 20 minutes.

While the various sizes of the compartments and the overall device housing may be varied, the size may generally vary depending on the variability of the packed RBC volume resulting from the washing process. For example, a unit draw may be typically between 450-500 mL (+/−10%), or more particularly 450 mL (+/−10%), which may result in a resulting volume of packed RBC cells of between 160-275 mL, or more particularly 200-250 mL of packed RBC cells. With this RBC volume, a wash solution volume including a RBC preservation solution and/or RBC processing solution may be introduced. In one example, about 110 mL of ADSOL® (Fresenius Kabi, Bad Hombur, Germany) or additive solution and about 50 mL of REJUVESOL® (Zimmer Biomet, Warsaw, Ind.) may be introduced into the device for washing of the RBC cells. Hence, the device may be sized to hold an approximate minimum total volume of about 370 mL (or less) and a maximum total volume of about 500 mL (or greater).

The device may be spun for a processing time of, e.g., 20 minutes or less, to achieve a 99% washout for any of the embodiments used spinning at, e.g., 4000 RPM or higher. To further increase the yield of the washed RBC cells, the surfaces of the device within may be coated or fabricated with hydrophobic surfaces and various geometries to increase the yield.

One factor for determining the number of cycles needed or desired to wash the RBC is the volume of waste remaining in the RBC wash compartment that may limit the efficiency of each wash cycle. Is it possible to result in a final volume that is exactly at a predetermined volume, e.g., 300 mL (red volume in 500 cc unit). The volume of the RBC wash compartment may be on the order of twice the volume of packed RBC so that there is about 50% to 65% hematocrit.

These parameters including the sizes, volumes, processing times, spin rates, etc. may be implemented to any one of the various embodiments described herein. Furthermore, any one of the variables may be altered to include parameters which are lower or greater depending upon the desired results and are still intended to be included within the scope of the disclosure.

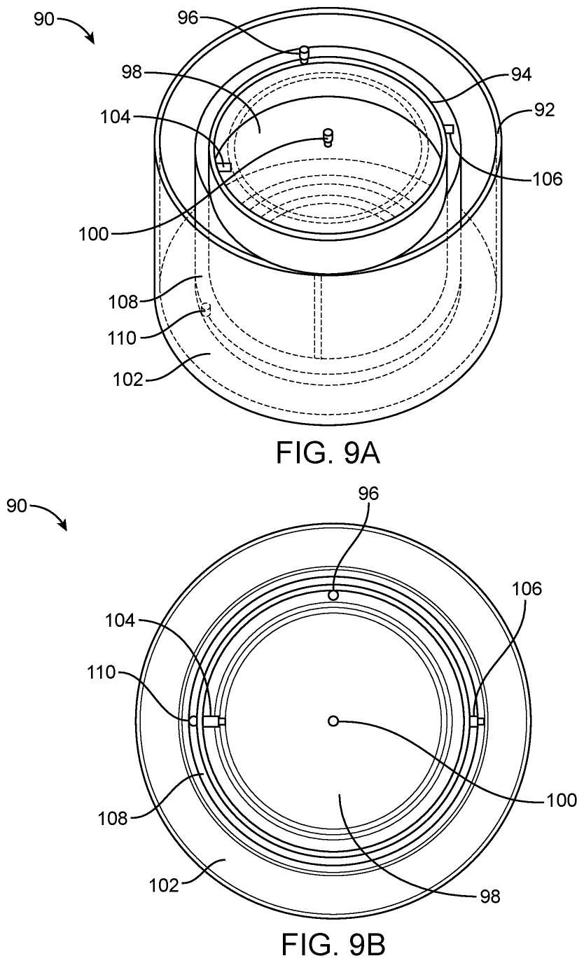

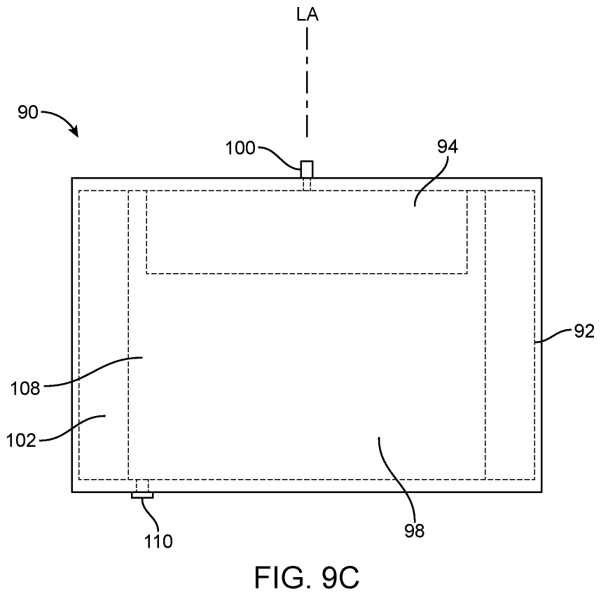

Turning now to one embodiment of a system which may be used to wash the RBC cells, FIGS. 9A to 9C illustrate perspective, top, and side views of one variation which uses a planetary red cell washer 90 which closely emulates the hand wash method in a fully automated process within a hermetically sealed rotor. The washer 90 may include a housing 92 illustrated in a cylinder configuration but which may be varied and which includes a RBC reservoir 94 within which is in fluid communication with an RBC inlet 96 positioned upon the top of the washer 90. A wash reservoir 98 may also be defined within the housing 92 and similarly in communication with a wash inlet 100. The washer 90 functions by diluting the RBC with a volume of the wash solution, sedimenting the RBC under centrifugation, and discarding the supernatant or waste solution. The process may be repeated one or several times to remove the desired amount of contaminating material from the RBC.

In this variation, the washer 90 may be comprised of three concentric compartments where the innermost compartment serves as the wash solution reservoir 98 and the outermost compartment serves as the waste reservoir 102 which collects the waste solution after washing the RBC volume. The intermediate compartment may serve as the RBC collection reservoir 108 which contains the washed RBC cells. The inner wash solution reservoir 98 and intermediate RBC collection reservoir 108 may communicate via a first valve 104 such as a centrifugal valve which may normally remain open connecting the two compartments at the top of the washer 90. At rest, though the first valve 104 may remain open, the wash solution and RBC are not mixed together because the open first valve 104 may remain above the fluid level. The intermediate RBC collection reservoir 108 and outer waste reservoir 102 may communicate via a second valve 106 such as a centrifugal valve which may normally remain closed, as shown in FIG. 9B.

When the rotor spins, the wash solution may rise at the outer boundary wall and spills through the open first valve 104 into the RBC collection reservoir 108. The solution may stop flowing into the RBC collection reservoir 108 when the compartment is full and the rotor may then ramp up to a higher speed to sediment the cells.

After a sufficient time to allow the cells to sediment, the centrifuge speed may be increased to generate sufficient force to close the normally open first valve 104, thereby isolating the RBC collection reservoir 108 from the wash solution reservoir 98. As the speed is further increased, the force may become sufficient to cause the outer centrifugal second valve 106 to open, permitting fluid to flow from the RBC collection reservoir 108 and into the outer waste reservoir 102. The flow path between these two compartments may be routed through a lumen extending from the outer wall of the RBC collection reservoir 108 up to a position within the RBC collection reservoir 108 which approximately coincides with the position of the interface between the packed cells and the supernatant wash waste so that only the latter drains out into the waste reservoir 102.

When the centrifuge ramps down, the first 104 and second 106 valves may return to their normal closed or open positions. As the rotor ramps down, fluid may begin to flow from the reservoir through the open valve into the RBC collection reservoir 108, which now once again contains only the packed cells. The second valve 106 between the RBC collection reservoir 108 and the waste reservoir 102 may return to its normally closed state so that the cells and new wash remain within the RBC collection reservoir 108.

The washer 90 can be optionally cycled to slightly increase and decrease speed, or reverse direction to ensure thorough mixing of cells with the wash solution. The washer 90 may again be ramped up to sediment the cells. If the RBC collection reservoir 108 has not yet filled with wash solution, additional wash solution may flow into the compartment until it is once again filled. The process sequence may continue through the completion of this second wash step, and if desired, the entire sequence can be repeated as many times as needed to reach the final desired RBC purity level.

The washer 90 of this variation may have an outer diameter of about 8 inches and a height of about 5 inches such that it has sufficient capacity to wash one unit of RBC up to, e.g., 4 times or more, with an equal volume of wash solution or it could be reconfigured to process, e.g., 2 washes with 2 volumes each. The wall extending from the ceiling to a position below the compartment floor may be seen and its radial position may correspond to the location of the lumen to ensure that the packed cells do not find their way into the drain line when waste is expelled.

The washer 90 may be removed from the centrifuge and the washed RBC cells may be drained through a RBC collection outlet 110 located along a bottom of the washer 90 for transfer to another container such as a collection bag, as shown in FIG. 9C.

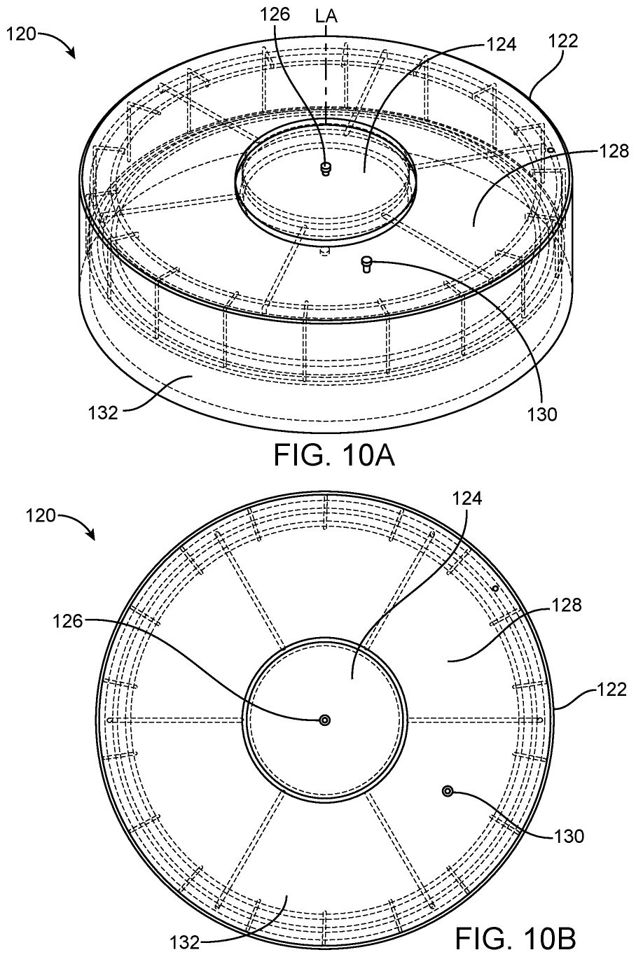

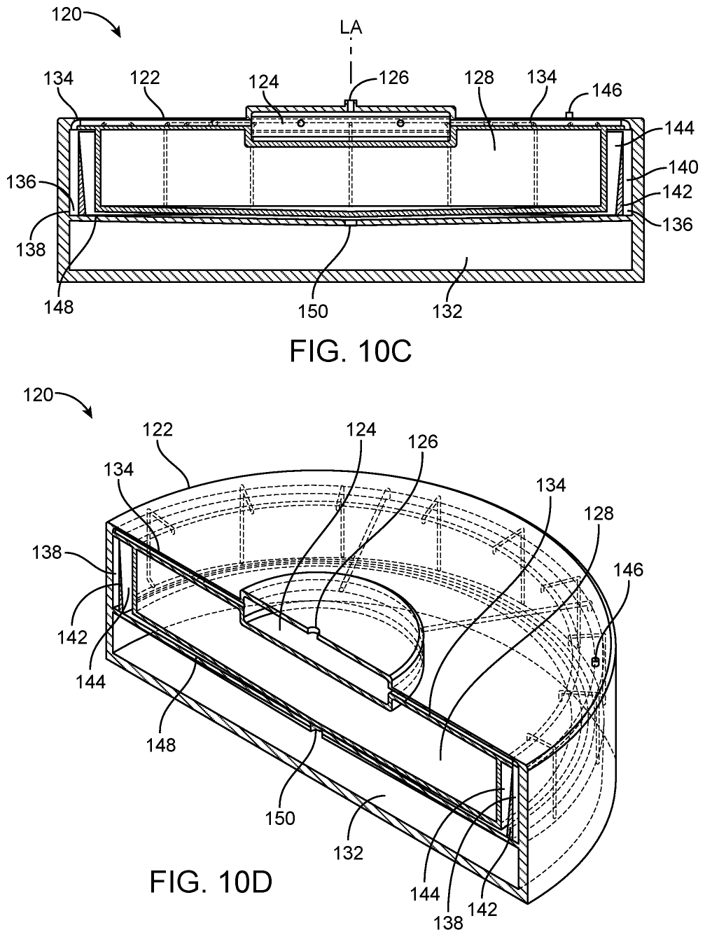

FIGS. 10A and 10B illustrate perspective and top views of another washer variation which utilizes centrifugation but eliminates the need for valves. Rather, the washer 120 relies on a geometry of the various compartments so that alternating centrifugal force and gravity alone directs fluid flow between the compartments. The washer 120 includes a housing 122 which is illustrated as a cylindrical structure but which may be configured in other shapes. A RBC reservoir 124 (cylindrically shaped) is located near or at the center of the housing 122 near the top of the housing 122 and is in fluid communication through a RBC inlet 126 into which a volume of blood may be introduced into the RBC reservoir 124. A wash reservoir 128 may be positioned below the RBC reservoir 124 and is also in fluid communication through a wash inlet 130 into which a volume of wash solution may be introduced. A waste reservoir 132 may be located below the wash reservoir 128. In this variation, the washer 120 diameter may be about, e.g., 1 ft and the height may be about, e.g., 3.5 inches, although these dimensions are presented as exemplary sizes and may be varied depending upon variations in the design of the washer 120.

FIGS. 10C and 10D illustrate cross-sectional and perspective cross-sectional views of the washer 120 to show the relative positioning of the various compartments. As shown, the RBC reservoir 124 is shown to be located atop or above the wash reservoir 128 which may be into a circularly configured compartment about which an overflow compartment 144 may be annularly arranged about an exterior of the wash reservoir 128. Adjacent to the overflow compartment 144 is a RBC wash compartment 138 annularly arranged about an exterior of the overflow compartment 144 such that a separating wall 142 (also annularly arranged) divides the overflow compartment 144 from the RBC wash compartment 138. The separating wall 142 may present an angled surface to the overflow compartment 144 such that the wall 142 is sloped or tapered relative to a longitudinal axis LA of the washer 120 where the angle tapers away from the overflow compartment 144 and towards the RBC wash compartment 138 the higher up along the separating wall 142.

One or more RBC feed lines 134 may be defined along the top of the housing 122 and extend radially towards the RBC wash compartment 138 such that the two compartments are fluidly coupled. One or more wash feed lines 136 may be defined through the housing 122 and extend radially from the wash reservoir 128 and open within a lower portion of the RBC wash compartment 138. A washed RBC outlet 146 may be defined along a top portion of the housing 122 above where the RBC wash compartment 138 is located such that any collected RBC pack 140 within may be withdrawn from the washer 120 upon completion of the washing process. A waste wash drainage gap 148 may be defined below the wash reservoir 128 and taper downwardly towards the center of the housing 122 towards a waste drainage opening 150 which opens to the waste reservoir 132 located below the wash reservoir 128.

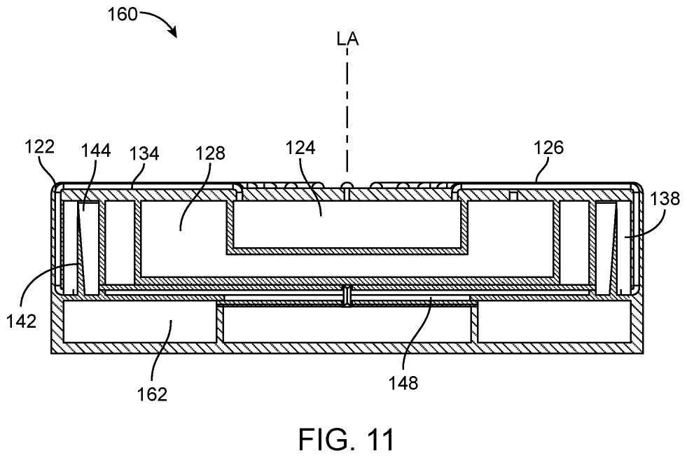

FIG. 11 illustrates a cross-sectional side view of another variation of a washer 160 which similarly incorporates the various compartments described, but in this variation, features like a sloped floor may be incorporated in the RBC wash compartment 138 to facilitate harvesting and/or one or more vanes may be incorporated, as described in further detail below, to minimize wave formation during ramp up and ramp down during centrifugation. To improve resuspension of the cells by wash solution percolating up through the bed, the configuration of the wash feed lines may be modified and the waste reservoir 162 may be separated into an annularly shaped compartment, as shown.

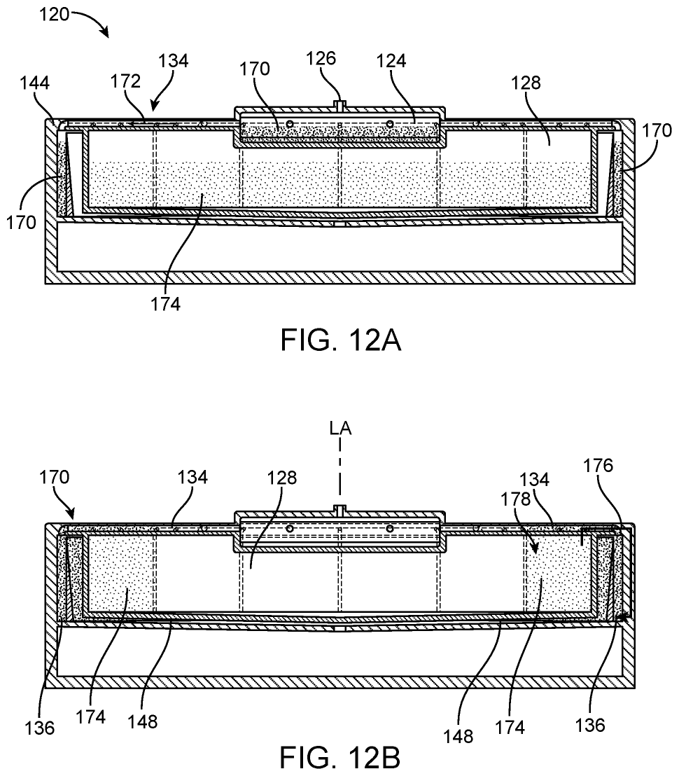

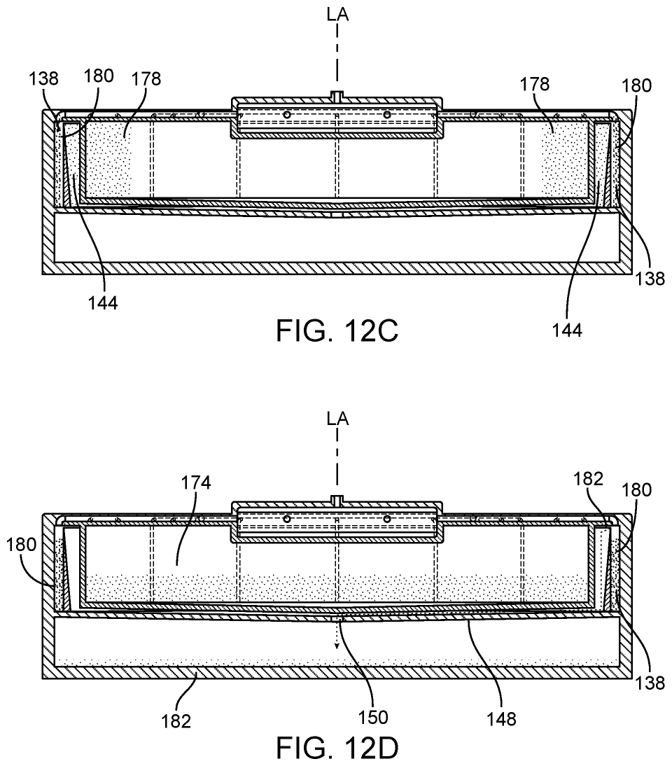

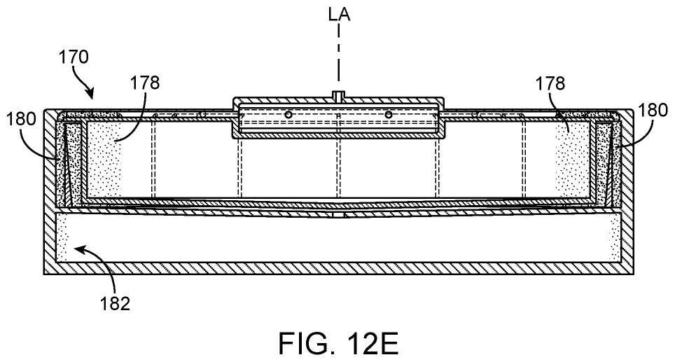

FIGS. 12A-12E illustrate one example of a sequence of steps that may be used in one method of the centrifugal process without the use of valves. Integral with the washer 120 can be an optional collection bag housed within a removable cap on the bottom of the washer 120 which may be isolated by a clamp on along a length of the connecting tubing to the RBC wash compartment 138. On completion of the wash process, the bag may be released and the clamp removed, allowing washed cells to drain into the bag, for example, from a bottom of the washer 120. This optional feature eliminates the need to perform a sterile connection for product recovery. Furthermore, the floor of the RBC containing compartment can optionally be sloped to one side to facilitate efficient product recovery.

In use, the washer 120 may be attached, coupled, or otherwise integrated with a motor or actuator for rotating the washer 120 to create the centrifugal force within. The wash solution 174 may be introduced into the wash reservoir 128, e.g., through the wash inlet 130. The total capacity of wash reservoir 128 may be varied, e.g., 2 liters of wash solution, and the compartment may be filled to below capacity so that under the force of centrifugation, a shell of wash solution is formed about the outer circumference of the wash reservoir 128 when centrifuged. A volume of RBC 170 is introduced through the RBC inlet 126 and into the RBC reservoir 124 such that some of the added RBC 170 may drain through one or more of the RBC feed lines 134, as indicated by the RBC flow 172, and into the RBC wash compartment 138. The RBC volume 170 in excess of the RBC reservoir 124 volume may remain in the lower portion of the RBC wash compartment 138 below the feed line entry openings.

As the washer 120 is spun about its longitudinal axis LA, the excess volume of RBC 170 within the RBC reservoir 124 may flow under centrifugal force through the RBC feed lines 134 and into the RBC wash compartment 138, as shown in FIG. 12B, and any volume in excess of the RBC wash compartment 138 capacity may spill over the top of the separating wall 142 into the overflow compartment 144 (interchangeably called a supernatant compartment). Simultaneously, wash solution 174 may be forced into a suspended state where the solution is pooled by the centrifugal force into the outer portion of the wash reservoir 128, as shown, such that wash solution 174 is flowed through the channels leading from the distal top of the wash solution reservoir 128 and into the bottom portion of the RBC wash compartment 138, as denoted by the arrow illustrating the wash solution flow 176. The wash solution 174 may percolate up through the RBC wash compartment 138 while mixing with the RBC volume contained within and spill over into the overflow compartment 144.

The fluid levels in the communicating compartments may equilibrate while under centrifugation limiting the volume of wash solution 174 which combines with the RBC 170 in the RBC wash compartment 138 and overflow compartment 144 (plus the small volume contained within the drain gap). Excess RBC 170 may spin out of the reservoir under centrifugation.

The inner side of the separating wall 142 dividing the RBC wash compartment 138 and overflow compartment 144 may be slightly angled, as described herein, so that under centrifugation any RBC contained within the overflow compartment 144 may slide up along the angled surface and out over the gap at the top of the separating wall 142 and into the RBC wash compartment 138. The centrifugal force may further prevent the suspension from draining out through the waste wash drainage gap 148 positioned below the wash reservoir 128. The combined volume of the RBC wash compartment 138 and overflow compartment 144 (as well as a small volume contained within the feed lines distal of the inner radius of the shell of fluid within the wash compartment) may define the total volume of RBC 170 plus wash solution 174 that can fill these compartments.

As shown in FIG. 12C, under continued centrifugation, an RBC pack 180 will sediment into the RBC wash compartment 138 and a small volume will also pack into the feedlines leading from the RBC reservoir 124. This negligible volume may remain in the feedlines throughout the processing. After the cells have sedimented, the washer 120 may be stopped and the waste wash 182 (supernatant) in the overflow compartment 144 may drain under the force of gravity through the waste drainage opening 150 and into the waste reservoir 132. In the absence of rotation, no further wash solution 174 may enter into the RBC wash compartment 138 as the feedlines communicate with the RBC wash compartment 138 via openings at the top of the RBC wash compartment 138, as shown in FIG. 12D.

Once the supernatant waste wash 182 has drained, the washer 120 may be spun again and fresh wash solution 174 may flow under centrifugal force from the wash reservoir 128 via the wash feedlines and into the bottom of the RBC pack 180 contained within the RBC wash compartment 138 where the wash solution 174 may again percolate up through the RBC pack 180 to again wash the RBCS, resuspending the cells and spilling out over the separating wall 142 and into the overflow compartment 144, as shown in FIG. 12E. Waste wash 182 may remain within the waste reservoir 132 as the drainage opening 150 is located at or near the rotational axis of the washer 120 and the pooled wash solution 178 may be retained within the wash reservoir 128 due to the centrifugation. The process may be repeated until the RBC wash compartment 138 and overflow compartment 144 are filled. Under continued centrifugation, the RBC pack 180 may form again and centrifugation may be stopped. The process may be repeated several times until the desired level of purity is attained. When completed, the washed RBC pack 180 may be collected from the RBC wash compartment 138. If desired, the solution may be agitated, for example, by brief abrupt start and/or stop pulses, to ensure sufficient resuspension before harvest. Slightly tilting the wash 120 or including a gradual slope of the floor of the RBC wash compartment 138 may also encourage the suspension to flow to one edge of the washer 120 to improve ease of harvest.

In this variation, the volume of the RBC wash compartment 138 may be, e.g., 250 mL, and the volume of the overflow compartment 144 may be, e.g., 300 mL. With a packed volume of, e.g., 200 mL of RBC, 50 mL (plus 6 mL of interstitial fluid) may remain outside the wall in the RBC wash compartment 138 when the 300 mL within the overflow compartment 144 is drained to waste. The extracellular solute remaining in the RBC wash compartment 138 may be thus about 16% of the starting amount. A total of, e.g., 4 washes may therefore yield about 99.9% removal. If the volume of RBC in the unit is less than, e.g., 200 mL, the washout may be a bit less, but an additional wash may be possible and may yield a high level of purity for any reasonable cell volume.

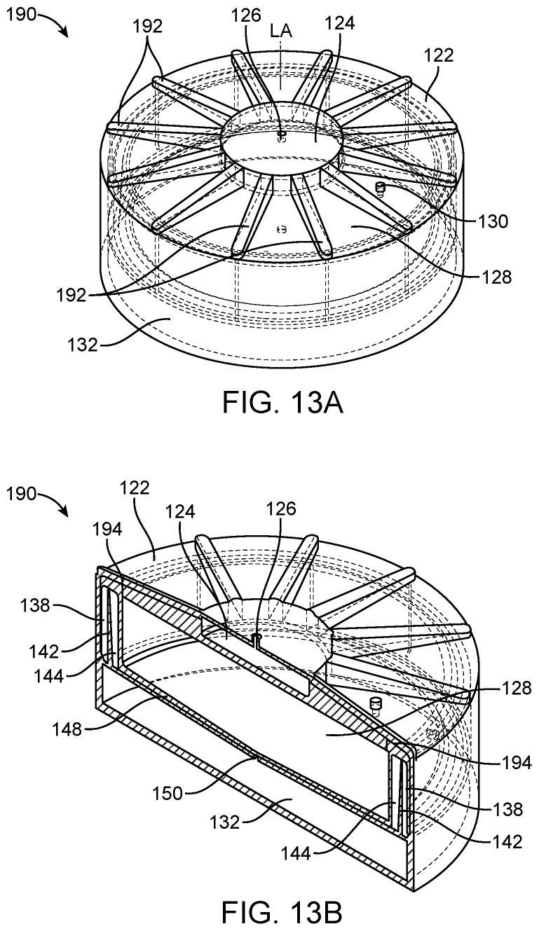

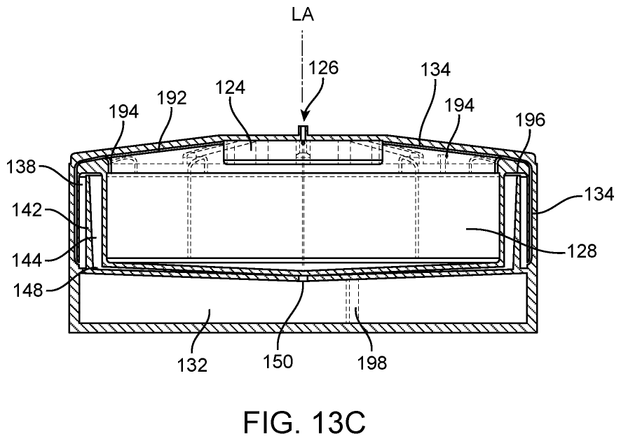

FIG. 13A illustrates a perspective view of yet another variation of the washer 190 where this variation includes one or more vanes 192 which are shown as elongate structures which emanate radially along the top of the housing 122 and include a wash feed line 194 through at least a portion of the vane 192. While the vanes 192 are shown in a uniform pattern symmetrically radiating about the housing 122, a single vane or a few vanes may be utilized in other variations.

FIGS. 13B and 13C illustrate cross-sectional perspective and cross-sectional side views of the washer 190 which illustrate how one or more of the vanes 192 may incorporate the wash feed line 194 through which the wash solution may be redirected through these channels within the vanes 192. Also shown are the spillover line 196 fluidly coupling the RBC wash compartment 138 and the overflow compartment 144 over the top of the separating wall 142. A collection port 198 may also be seen fluidly coupling the RBC wash compartment 138 for optionally collecting the RBC pack from the bottom of the washer 190.

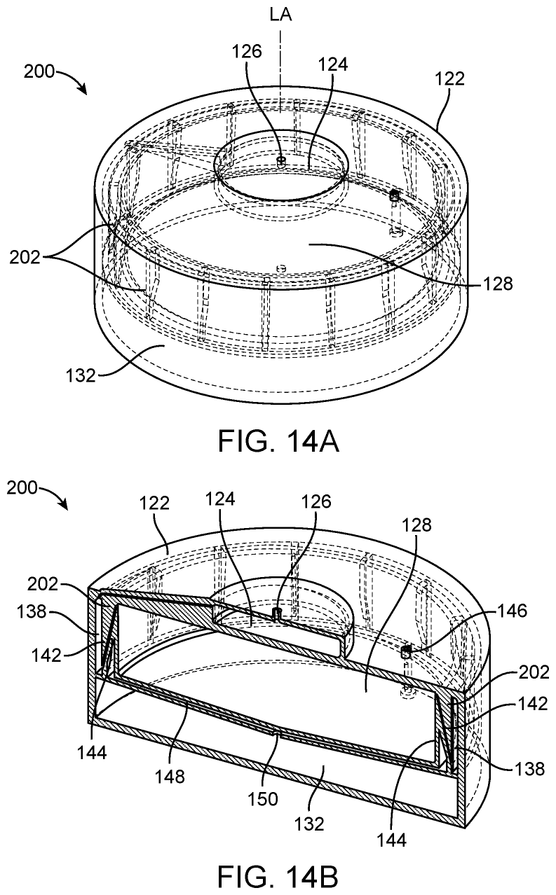

FIGS. 14A and 14B show perspective and cross-sectional perspective views of yet another variation of the washer 200. In this variation, one or more vanes 202 may be situated within or along the overflow compartment 144 and may serve to minimize wave formation in the solution during centrifugation ramp up and ramp down. The one or more vanes 202 may also include channels through which the wash solution may be directed to the bottom of the RBC wash compartment 138.

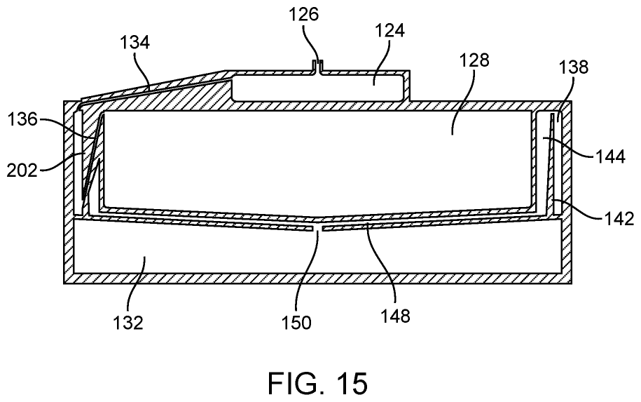

FIG. 15 illustrates a cross-sectional side view of the embodiment of FIGS. 14A and 14B. As shown, the vane 202 may be formed internally so that the wash feed lines 136 may extend from the top of the wash reservoir 128 and through the vane 202 and into the bottom of the RBC wash compartment 138. As the vane 202 allows for the passage of fluid through the vane 202, the flow of the wash solution and/or RBC into or out of the overflow compartment 144 may be slowed or dampened as the washer is spun up or slowed down to control the flow.

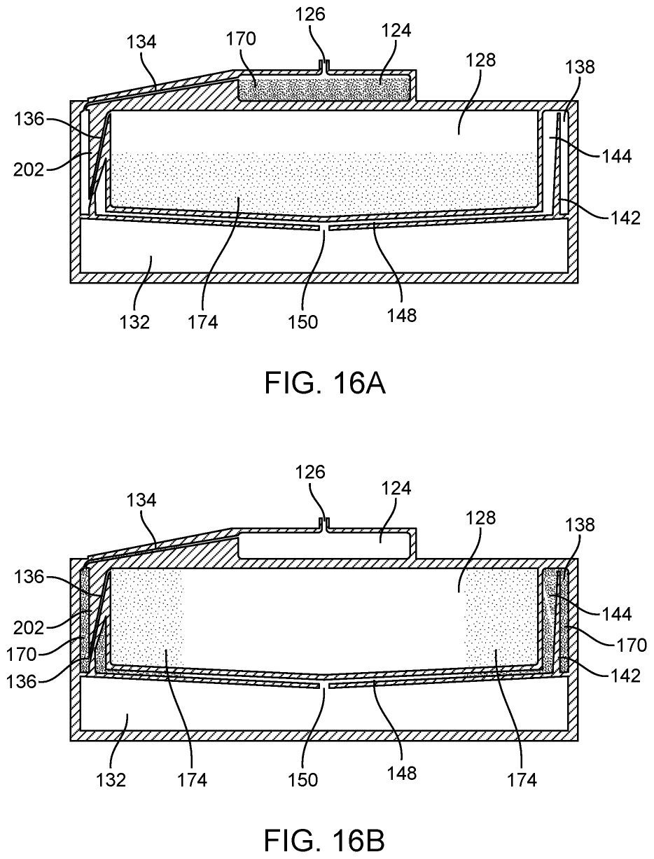

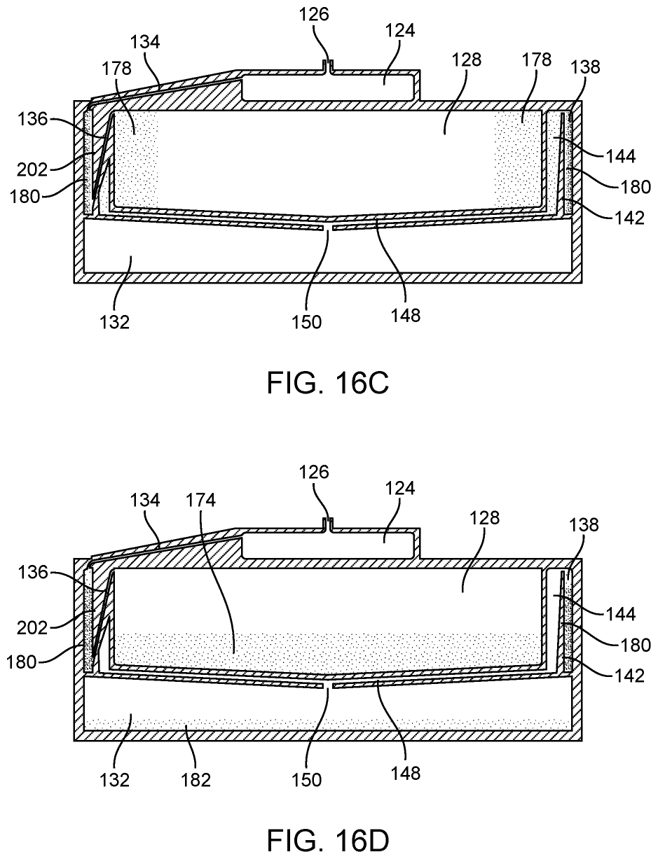

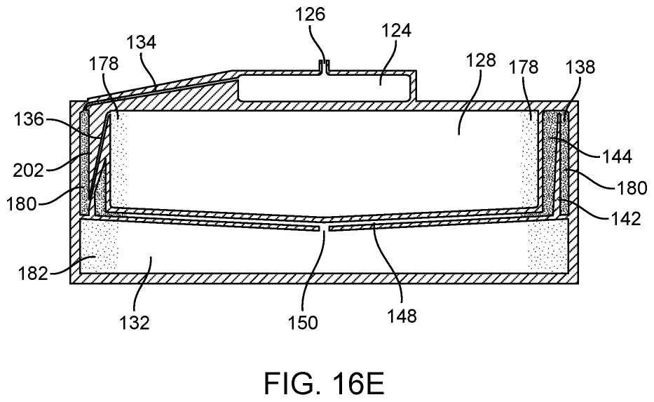

FIGS. 16A to 16E show cross-sectional side views illustrating one variation of how the RBC 170 may be introduced into the RBC reservoir 124 and the wash solution 174 into the wash solution reservoir 128. The washer may spin about its longitudinal axis LA and the RBC 170 may flow from the RBC reservoir 124, through the RBC feedline 134 which may be angled relative to the longitudinal axis LA, and into the top of the RBC wash compartment 138 which may be formed circumferentially to form the annular compartment, as shown in FIG. 16B. The wash solution 174 may flow from the wash solution reservoir 128, through the wash solution feedline 136, and into the bottom of the RBC wash compartment 138. The wash solution feedline 136 may also be angled relative to the longitudinal axis LA and may flow into the bottom or lower portion of the RBC wash compartment 138.

With continued centrifugation, the RBC may sediment into the RBC wash compartment 138 as the wash solution 178 percolates upwardly through the RBC wash compartment 138 and flows into the overflow compartment 144 while leaving the RBC pack 180 within the RBC wash compartment 138, as shown in FIG. 16C. The slope on the inner surface of the separating wall 142 allows for the RBC to climb up the inner surface and escape through the gap or channel at the top of the separating wall 142 into the RBC wash compartment 138. The wash solution 178 may also be seen pooled or suspended around the inner wall of the wash reservoir 128 due to the centrifugation.

When the washer stops spinning, the RBC may slump down and remain isolated within the RBC wash compartment 138, as shown in FIG. 16D. The waste wash 182 in the overflow compartment 144 may drain out through the drainage opening 150 via the drainage gap 148 and into the waste reservoir 132.

When the washer spins again, additional wash solution 178 may again flow into the bottom of the RBC wash compartment 138, percolating up through the RBC pack 180, as shown in FIG. 16E, to further wash the RBC contained within. The wash waste 182 may remain isolated within the waste reservoir 132. The sequence of steps may be repeated one or several times to achieve sufficient removal of extracellular solutes. For example, the wash reservoir may accommodate enough volume for up to four washes although fewer than four or greater than four washes may be performed.

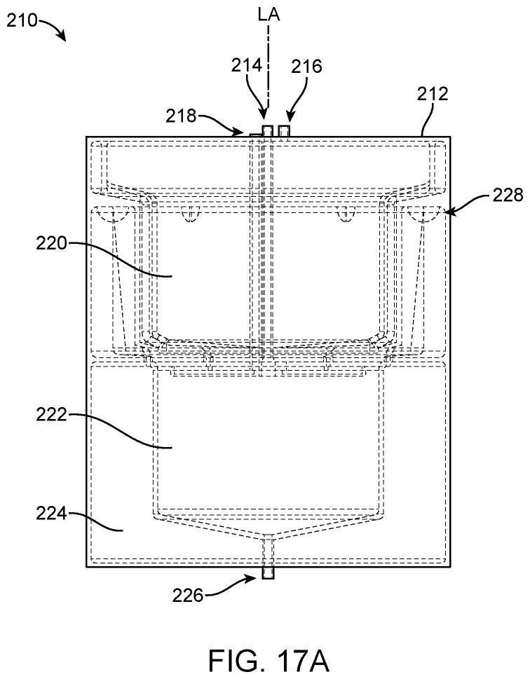

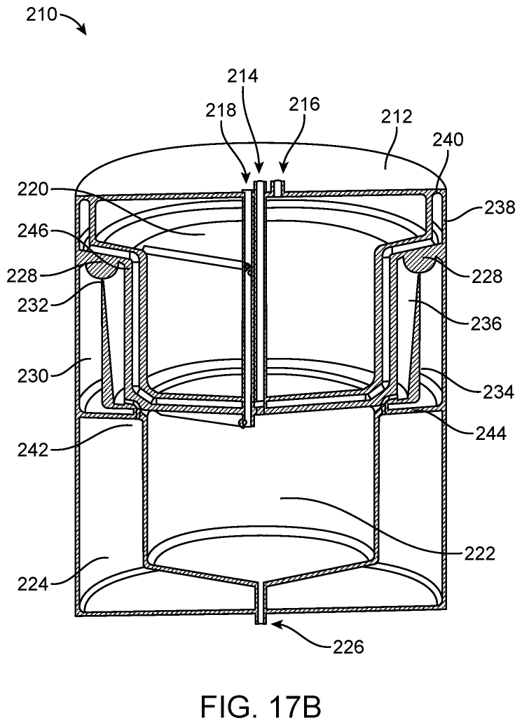

Yet another variation of the washer 210 is shown in the side and cross-sectional perspective views of FIGS. 17A and 17B. This variation of the washer 210 may include a housing 212 upon which a RBC inlet 214 positioned upon a top portion of the housing 212 may be fluidly coupled to a RBC wash compartment 230. A wash inlet 216 may be also be positioned upon the top portion of the housing 212 and may be fluid coupled to a wash reservoir 220 within the housing 212. A vent 218 may be in fluid communication with each compartment within the housing 212. A receiver cup 222 may be defined within the housing 212 below the wash reservoir 220 and the wash waste reservoir 224 may be defined annularly about the receiver cup 222. A washed RBC collection port 226 may be integrated along a bottom portion of the receiver cup 222 and optionally coupled to a removable collection reservoir or may be capped via a removable covering for collecting the RBC pack formed within the receiver cup 222.

The washer 210 may be loaded with a unit of RBC and, e.g., 1 liter of wash solution, introduced though a tubing set optionally integrated with the washer 210. After introduction, the tubing be removed or otherwise sealed. The receiver cup 222 has the capacity to contain an RBC unit volume, e.g., of up to 505 mL or greater, and the washer 210 may be spun and stopped any number of times, e.g., 8 times to achieve 8 batch washes, in order to yield a washout of greater than 99.6% of solutes from a unit containing, e.g., 170 mL, of packed RBC in a total volume of, e.g., 370 mL.

In this variation, the receiver cup 222 may function to collect the washed RBC pack after washing within the RBC wash compartment. The wash solution within the wash reservoir 220 may flow under centrifugation through the opening 240, through the metering chamber 238, and into the RBC wash compartment 230 which is similarly positioned annularly relative to the overflow compartment 236 while separated by the separating wall 234. The two compartments may remain in fluid communication via the gap 232 for RBC spillover. Once centrifugation has been stopped, the washed RBC pack may flow from the RBC wash compartment 230 and into the receiver cup 222 via a gap or channel 244 defined to be in fluid communication between the two compartments. In the manner, the washed RBC may be collected directly from the receiver cup 222. The waste wash solution may be drained into the waste reservoir through opening 242 for waste drainage. The metered wash solution may further flow through channels 246 from the metering chamber 238 via an RBC input feedline. Furthermore, one or more vanes 228 may be incorporated internally within the reservoirs to minimize wave formation during ramp up and ramp down and to also prevent spillover during centrifugation ramp down. Because the sedimentation depth is small, the duration of each spin/stop cycle may range between about, e.g., 2-3 minutes, for a total process time on the order of 20 minutes or less. This may also keep hemolysis at a minimum.

Upon completion of a washing process, the washed RBC within the receiver cup 222 may be removed via the RBC collection port 226 and through an optional integral tubing set connected to the collection port 226 on the bottom of the washer 210. The tubing set may be optionally housed within a detachable cap during processing and the tubing set can be used to administer washed RBC directly from the washer 210 and to the patient, for example, with the washer 210 removed from a centrifuge base unit and suspended from an IV pole. Optionally, the tubing can be sterile docked to a bag and the washed RBC drained into the bag for future use.

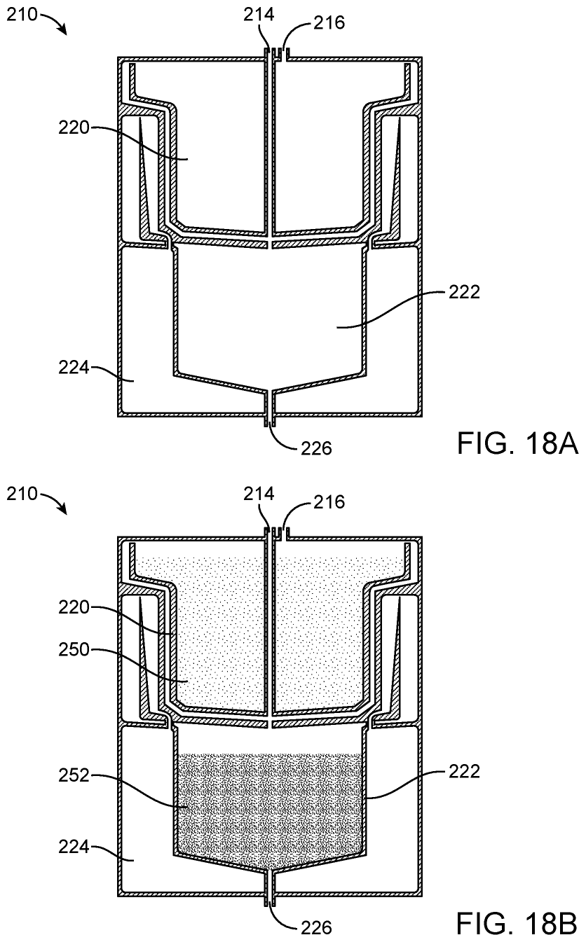

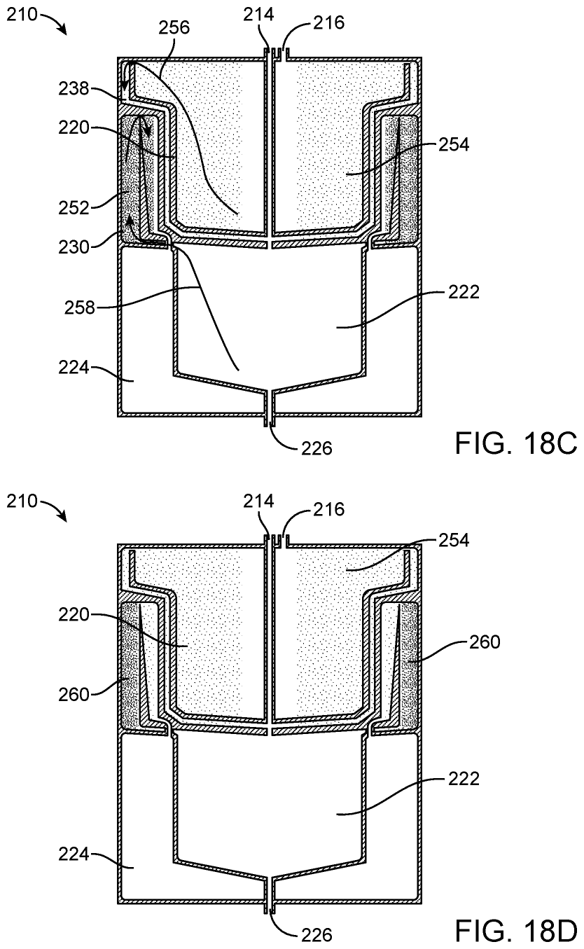

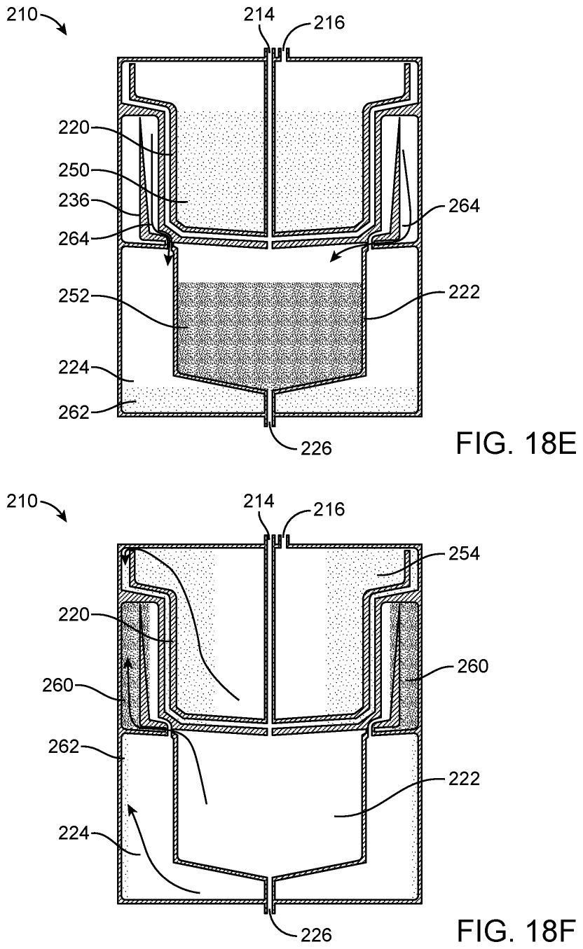

FIGS. 18A to 18F illustrate schematic side views of one method for how the washer 210 may process a volume of the RBC during centrifugation. FIG. 18A illustrates the interior cross-sectional side view of the washer 210 ready to load. FIG. 18B illustrates how a volume of wash solution 250 may be introduced through the wash inlet 216 to at least partially fill the wash reservoir 220 and a volume of RBC 252 may be introduced through the RBC inlet 214 to at least partially file the receive cup 222.

As the washer 210 begins spinning about its longitudinal axis LA, the RBC may begin to flow 258 from the receiver cup 222 and up into the RBC wash compartment 230 as the wash compartment 230 is positioned further radially from the longitudinal axis LA than the receiver cup 222, as shown in FIG. 18C. Likewise, the wash solution 254 may become suspended around the inner walls of the wash reservoir such that the wash solution flows 256 into the metering chamber 238 and into contact with the RBC 252, as shown in FIG. 18D. There may be a time lag in the fluids fully displacing within the housing from when the washer 210 begins spinning and when the fluids are fully suspended due to the centrifugal force.

As the washer 210 is slowed or stopped, the waste wash may flow 264 from the overflow compartment 236 and directly into the waste reservoir 224 and the washed RBC 260 may flow from the RBC wash compartment 230 back into the receiver cup 222, as shown in FIG. 18E. The washing sequence may be repeated, as shown in FIG. 18F, until the wash solution 254 is depleted and RBC pack 260 is clean. At completion, the washer 210 may be started/stopped one or more times to completely suspend the RBC such that the final volume of washed RBC is back within the receiver cup 222. The final amount of hematocrit may be variable (e.g., from 52% to 85%) depending upon the RBC input hematocrit and volume.

The depth of the suspension within the wash station determines the amount of time required to sediment the RBC. With a shallower and wider profile, the cells have less distance to travel and may therefore sediment in a shorter time. However, if the space is taller and narrower, the viscous slurry of cells may not drain as well when the washer stops spinning (particularly for units with high volume of packed RBC). The sedimentation distance in the washer may range anywhere from, e.g., about 1.5 cm, and the depth of the resuspended cell slurry distal to the isolation wall may be, e.g., about 1.0 cm.

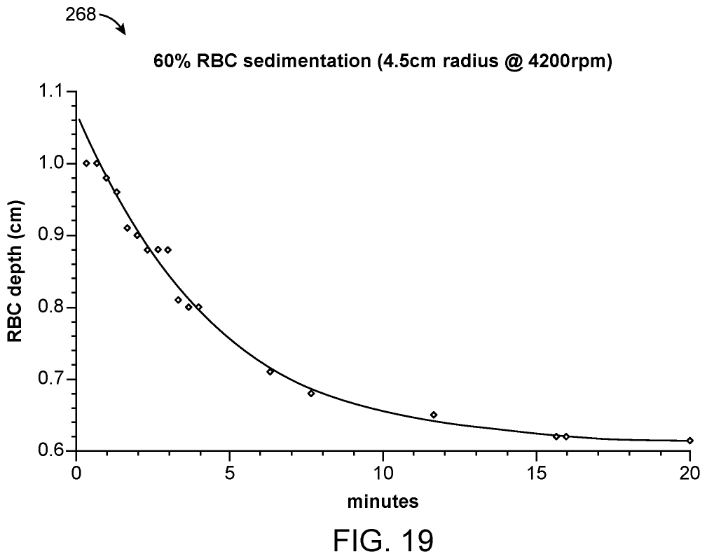

FIG. 19 illustrates a graph 268 showing an example of RBC sedimentation of RBC depth over time. Sedimentation to, e.g., about 7.5 mm, may be sufficient to pack the cells comfortably behind the wall in a washer at this relative centrifugal force (RCF) at 1.1 cm suspension depth. By varying reducing factors such as the height of the washer, the sedimentation depth may be decreased to proportionately decrease the wash cycle time. Hence, the wash cycle times may be reduced down to about 2 minutes each (including ramp up and ramp down) for a total process time of around 20 minutes or less.

The RBC pack recovered from the RBC wash compartment may be resuspended. In one example, the suspension was spun in a clinical centrifuge to resediment the RBC and the recovered volume was about 23 mL at a hematocrit level of about 85%. This represented a 92% recovery of the annular compartment volume of 25 mL.

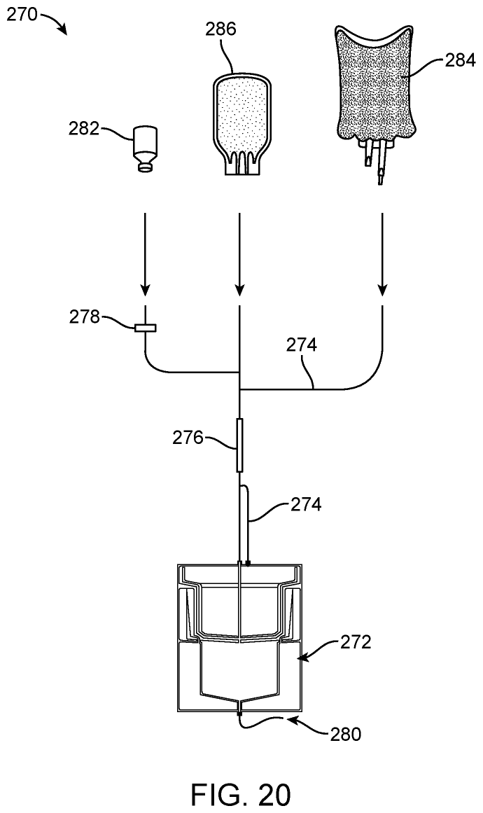

The washer itself may be included into a complete system or assembly which may be provided to users. One example of such an assembly 270 is shown in the schematic of FIG. 20 which illustrates a disposable collection tubing set 280 (which may integral with the centrifugal washer 272). A tubing set 274 may also be included for fluidly coupling to the appropriate inlet ports of the washer 272 which suitable tubing for fluidly coupling to, e.g., a first solution 282 such as Rejuvesol®, a second solution 284 such as a wash solution, and to a volume of collected RBC 286. An optional filter or flow restrictor 278 may be integrated or included for use in any of the tubing in tubing set 274 as well as an optional heat exchanger 276 which may be incorporated along one or more portions of the tubing.

The wash solution 284 and RBC 286 may be sterile docked to tubing 274 attached to washer 272. The first solution 282 may be coupled via puncture of the vial septum and the filter or flow restrictor 278 may control the feed of the various inputs via the heat exchanger 276 into the washer 272, which may be housed in a warmed centrifuge. After filling the washer 272, the tubing set 274 may sealed and severed near the washer 272 by a sterile docking mechanism integral with the base unit. After incubation, the washer 272 may process one or more wash cycles and upon completion, the collection tubing set 280 may be used to collect the washed RBC.

The first solution 282 and RBC 286 introduced to the washer 272 via the attached tubing 274 via a heat exchanger 276 which may warm them to, e.g., 37 degrees, and incubation may be carried out within the washer 272 with gentle mixing provided by starting and stopping the centrifuge several times during incubation. The centrifuge may be warmed so as not to draw heat out of the sample during processing.

The apparatus and methods disclosed above are not limited to the individual embodiments which are shown or described but may include combinations which incorporate individual features between the different variations. Modification of the above-described assemblies and methods for carrying out the invention, combinations between different variations as practicable, and variations of aspects of the invention that are obvious to those of skill in the art are intended to be within the scope of the claims.

What is claimed is:

1. An apparatus for centrifuging a suspension of red blood cells, comprising:
a reservoir positioned within a housing and located along an axis of rotation, wherein the reservoir defines a port for receiving the suspension of red blood cells;
a wash reservoir located below around the reservoir;
a pack compartment located circumferentially about the reservoir and in fluid communication with the wash reservoir through a fluid lumen extending from a top portion of the wash reservoir and a bottom portion of the pack compartment;
a supernatant compartment in fluid communication with the pack compartment and located circumferentially between the wash reservoir and the pack compartment; and
a waste reservoir located below the wash reservoir and in fluid communication with the supernatant compartment.

2. The apparatus of claim 1 wherein the reservoir defines an excess red blood cell hold region.

3. The apparatus of claim 1 further comprising a valve positioned along the top portion of the wash reservoir.

4. The apparatus of claim 1 wherein the supernatant compartment comprises a wall having a surface angled relative to the axis of rotation.

5. The apparatus of claim 1 wherein the supernatant compartment is in fluid communication with the waste reservoir via a fluid lumen.

6. A method for centrifuging a suspension of red blood cells, comprising:
introducing the suspension of red blood cells within a reservoir such that the suspension drains from the reservoir and into a pack compartment circumferentially positioned about the reservoir;
rotating the suspension within the circumferential pack compartment such that a wash solution within a wash compartment flows radially outward and up through the pack compartment and into a supernatant compartment; and
stopping a rotation of the suspension such that waste from the supernatant compartment drains into a waste compartment and a RBC pack remains within the pack compartment.

7. The method of claim 6 further comprising repeating each of the steps from one to four times.

8. The method of claim 6 wherein introducing the suspension comprises introducing the red blood cells within the reservoir located along an axis of rotation of the reservoir.

9. The method of claim 6 wherein rotating the suspension comprises flowing the wash solution through one or more openings which flow into a bottom of the pack compartment.

10. The method of claim 6 wherein the supernatant compartment is located circumferentially within the pack compartment.

11. The method of claim 6 wherein the waste reservoir is located circumferentially below the wash compartment.

12. A method of filtering a suspension of red blood cells, comprising:
introducing a wash solution to the suspension of red blood cells such that a combined volume is retained within a first compartment;
applying a sweeping action upon the volume;
applying a pressure upon the volume while applying the sweeping action such that the volume is urged through a filter membrane at a predetermined filtration rate;
maintaining the filtration rate over a predetermined period of time such that hemolysis is maintained below a threshold level; and
collecting the volume within a second compartment.

13. The method of claim 12 wherein introducing the wash solution comprises introducing the wash solution such that the volume has 10% hematocrit level.

14. The method of claim 12 wherein applying the sweeping action comprises passing the volume over a polycarbonate or polyethylene track etched membrane.

15. The method of claim 12 wherein applying the pressure comprises applying 0.5 to psi of pressure upon volume.

16. The method of claim 12 wherein applying the pressure further comprises urging the volume through the filter membrane having a pore size of 0.8 to 1.2 micron.

17. The method of claim 12 wherein maintaining the filtration rate comprises maintaining the filtration rate for less than 30 minutes to 1 hour.

18. The method of claim 12 wherein maintaining the filtration rate comprises maintaining the hemolysis threshold level below 1%.

19. The method of claim 12 wherein maintaining the filtration rate comprises removing extracellular solutes from the volume.

20. The method of claim 19 wherein removing extracellular solutes comprises removing over 99% of the extracellular solutes from the volume.

21. The method of claim 12 further comprising repeating each of the steps from one to four times.
